(19) United States
(12) Patent Application Publication
(10) Pub. No.: US 2023/0123889 A1
(43) Pub. Date: Apr. 20, 2023

(54) METHOD FOR DEGRADING BENZO(A)PYRENE WITH A HALOPHILIC BACTERIUM STRAIN OF STAPHYLOCOCCUS HAEMOLITICUS

(71) Applicant: KING FAHD UNIVERSITY OF PETROLEUM AND MINERALS, Dhahran (SA)

(72) Inventors: Alexis NZILA, Dhahran (SA); Musa Mohammed Musa MUSA, Dhahran (SA); Saravanan SANKARA, Dhahran (SA)

(73) Assignee: KING FAHD UNIVERSITY OF PETROLEUM AND MINERALS, Dhahran (SA)

(21) Appl. No.: 17/505,060

(22) Filed: Oct. 19, 2021

(51) Int. Cl. C02F 3/34; C12N 1/20

(52) U.S. Cl. CPC C02F 3/34; C12N 1/205; C12R 2001/44

(57) ABSTRACT

A method for degrading a polycyclic aromatic hydrocarbon such as the 5-membered ring compound benzo(a)pyrene (BZP) using a halophilic microbe Staphylococcus haemoliticus, strain 10SBZ1A.

### REFERENCE TO A SEQUENCE LISTING

In accordance with 37 CFR § 1.52(e)(5), the present specification makes reference to a Sequence Listing which is submitted electronically as a .txt file named “527132US_Sequence_Listing_ST25.txt”. The .txt file was generated on Feb. 19, 2020 and is 100 kb in size. The entire contents of the Sequence Listing are herein incorporated by reference.

### Statement Regarding Prior Disclosures by the Inventor(s)

A partial rDNA sequence for strain Staphylococcus haemolyticus strain 10SBZ1A has been submitted on Aug. 29, 2019 to GENBANK under NCBI accession number GI: MN388897.

### BACKGROUND OF THE INVENTION

### Field of the Invention

The invention falls within the fields of microbiology and environmental chemistry and involves the use of a newly isolated halophilic Staphylococcus haemolyticus strain 10SBZ1A to degrade and bioremediate polycyclic aromatic hydrocarbons (PAHs) such as benzo(a)pyrene (BZP).

### Description of the Related Art

Some of the most important pollutants found in petroleum products are polycyclic aromatic hydrocarbon (PAHs). They include several groups of ringed compounds such as those having 2-rings like naphthalene (NAPH); 3-rings like phenanthrene (PHEN) and anthracene (ANTH); 4 rings like pyrene (PYR) and 5-rings, such as benzo(a)pyrene (BZP).

PYR and BZP are considered high-molecular weight PAHs (HMW-PAHs) which are relatively difficult to degrade. The biodegradation of PAHs is challenging as difficulty in biodegradation increases with ring number. BZP, because of its high ring number, is one of the most resistant to degradation and as a result it persists longer in the environment with its attendant toxicity. BZP is ranked as number 8 out of 275 chemicals on the priority list of hazardous substances for Comprehensive Environmental Response, Compensation, and Liability Act (CERCLA); see hypertext transfer protocol secure://www.epa.gov/enforcement/comprehensive-environmental-response-compensation-and-liability-act-cercla-and-federal (last accessed Feb. 13, 2020) and the priority list of hazardous substances that will be the subject of toxicological profiles, hypertext transfer protocol secure://www.atsdr.cdc.gov/SPL/index.html.

BZP is toxic to both marine flora and humans, is carcinogenic and exhibits developmental, neurological, reproductive and immunological toxicities; Bostrom, C E, et al. (2002). Cancer risk assessment, indicators, and guidelines for polycyclic aromatic hydrocarbons in the ambient air. Environ Health Perspect 110 Suppl 3: 451-488; and Ramesh, A and AE Archibong (2011). Chapter 43—Reproductive toxicity of polycyclic aromatic hydrocarbons: occupational relevance. Reproductive and Developmental Toxicology. RC Gupta. San Diego, Academic Press: 577-591. The removal of this pollutant from the environment remains a priority.

The degradation of PAHs by active non-halophilic microorganisms has been studied extensively in the past two decades; Kanaly, R A and S Harayama (2000). Biodegradation of high-molecular-weight polycyclic aromatic hydrocarbons by bacteria. J Bacteriol 182(8): 2059-2067; Seo, J-S, Yet al. (2010). Degradation of pyrene by Mycobacterium aromativorans strain JS19b1. Journal of the Korean Society for Applied Biological Chemistry 53(3): 323-329; Nzila, A (2013). Update on the cometabolism of organic pollutants by bacteria. Environ Pollut 178: 474-482; Budiyanto, F, et al. (2017). Characterization of Halophilic Bacteria Capable of Efficiently Biodegrading the High-Molecular-Weight Polycyclic Aromatic Hydrocarbon Pyrene. Environmental Engineering Science 35(6): e; Nzila, A, et al. (2017). Isolation and characterisation of bacteria degrading polycyclic aromatic hydrocarbons: phenanthrene and anthracene. Arch Environ Prot 44(2): 43-54 and Nzila, A (2018). Current Status of the Degradation of Aliphatic and Aromatic Petroleum Hydrocarbons by Thermophilic Microbes and Future Perspectives. Int J Environ Res Public Health 15(12).

Although most of this work focused on PAHs containing 2-4 rings, a few microbes were reported to degrade BZP under non-halophilic conditions. These include Sphingomonas yanoikuyae JA, Mycobacterium sp., Mycobacterium vanbaalenii, Stenotrophomonas maltophilia, Novosphingobium pentaromativorans, Mesoflavibacter zeaxanthinfaciens, Ochrobactrum sp., Bacillus μlichenformis and Bacillus subtilis; see Sohn, J H, K K Kwon, et al. (2004). Novosphingobium pentaromativorans sp. nov., a high-molecular-mass polycyclic aromatic hydrocarbon-degrading bacterium isolated from estuarine sediment. Int J Syst Evol Microbiol 54(Pt 5): 1483-1487; Moody, J D, J P Freeman, et al. (2005). Degradation of benz[a]anthracene by Mycobacterium vanbaalenii strain PYR-1. Biodegradation 16(6): 513-526; Husain, S (2008). Literature Overview: Microbial Metabolism of High Molecular Weight Polycyclic Aromatic Hydrocarbons. Remediation Journal 18(2): 131-161; Rentz, J A, P J Alvarez, et al. (2008). Benzo[a]pyrene degradation by Sphingomonas yanoikuyae JAR02. Environ Pollut 151(3): 669-677; Lily, M K, A Bahuguna, et al. (2009). Degradation of Benzo [a] Pyrene by a novel strain Bacillus subtilis BMT4i (MTCC 9447). Braz J Microbiol 40(4): 884-892; Wu, Y, T He, et al. (2009). Isolation of marine benzo[a]pyrene-degrading Ochrobactrum sp. BAP5 and proteins characterization. Journal of Environmental Sciences 21(10): 1446-1451; Chen, S, H Yin, et al. (2013). Effect of copper(H) on biodegradation of benzo[a]pyrene by Stenotrophomonas maltophilia. Chemosphere 90(6): 1811-1820; Okai, M, I Kihara, et al. (2015). Isolation and characterization of benzo[a]pyrene-degrading bacteria from the Tokyo Bay area and Tama River in Japan. FEMS Microbiol Lett 362(18): fnv143; and Guevara-Luna, J, P Alvarez-Fitz, et al. (2018). Biotransformation of benzo[a]pyrene by the thermophilic bacterium Bacillus licheriformis M2-7. World J Microbiol Biotechnol 34(7): 88.

The degradation of PAHs containing 2-4 rings has also been reported in salinity conditions by various halophilic microbes; see Margesin, R and F Schinner (2001). Biodegradation and bioremediation of hydrocarbons in extreme environments. Appl Microbiol Biotechnol 56(5-6): 650-663; Martins, L F and R S Peixoto (2012). Biodegradation of petroleum hydrocarbons in hypersaline environments. Braz J Microbiol 43(3): 865-872; and Fathepure (2014). Aruzahgan et al. reported a stain of Ochrobactrum sp.VP1 capable of using BZP as the sole source of carbon under moderate salinity conditions of NaCl 3% (Arulazhagan and Vasudevan 2011). See Arulazhagan, P and N Vasudevan (2011). Biodegradation of polycyclic aromatic hydrocarbons by a halotolerant bacterial strain Ochrobactrum sp. VA1. Mar Pollut Bull 62(2): 388-394.

A consortium of bacteria (Achromobacter sp. AYS3, Marinobacter sp. AYS4 and Rhodanobacter sp. AYS5) has also been reported to degrade BZP in the presence of PHEN, in the same salinity conditions (NaCl 3%). See Arulazhagan, P, C Sivaraman, et al. (2014). Co-metabolic degradation of benzo(e)pyrene by halophilic bacterial consortium at different saline conditions. Journal of Environmental Biology 35: 445-452. So far, only one BZP-degrading strain, Ochrobactrum sp.VP1, has been shown to utilize BZP as a single strain, but at relatively low salinity of only 3%.

Accordingly, to address the shortcomings of prior methods, a method of degrading BZP with a halophilic bacterium in 10% NaCl is provided together with the halophilic bacterium. The capacity of this bacterium for removing BZP in contaminated soil samples is shown. This microorganism provides a convenient and efficient way to degrade PAHs, such as BZP, in fluids or soils having substantial salt content.

### SUMMARY OF THE INVENTION

One aspect of the invention is a method for removing a PAH, such as BZP, from a contaminated fluid or solid, such as soil or solid waste by contacting the contaminated material with Staphylococcus haemolyticus strain 10SBZ1.

In one embodiment, this method is performed in a fluid or solid that contains at least 5%, 10% or 15% wt/wt of NaCl.

In another embodiment, this method is performed in a soil contaminated with a PAH, such as BZP, wherein the soil may also contain at least 5 to 15% (wt/wt) of NaCl.

In another embodiment, this method is performed in a fluid contaminated with a PAH, such as BZP, wherein the fluid may also contain at least 5 to 15% (wt/wt) of NaCl.

In another embodiment, this method is performed in a contaminated fluid or solid in which the PAH is substantially the sole carbon source for Staphylococcus haemolyticus strain 10SBZ1.

Another aspect of the invention is a halophilic microorganism derived from Staphylococcus haemolyticus strain 10SBZ1A that degrades PAHs such as BZP.

In some embodiments, this halophilic microorganism will have 16s rDNA that is at least 99% identical to that of Staphylococcus haemolyticus strain 10SBZ1A.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1A is an electron micrograph showing Gram-negative bacteria that are coccus-shaped with a diameter of about 1.2 μM.

FIG. 1B shows the effect of BZP on bacterial growth.

FIG. 2 depicts an increase in bacterial culture doubling time (dt) as the BZP concentration increases.

FIG. 3 shows that the ability of bacterial strain 10SBZ1A to metabolize or degrade PAHs increases as the PAH ring number decreases as measured by differences in doubling time.

FIG. 4 shows the degradation profile BZP by bacterial strain 10SBZ1A.

FIG. 5 compares the rate of BZP degradation by 10SBZ1A to other strains which are not halophilic.

FIG. 6 depicts potential degradation pathways.

FIG. 7 depicts some PAHs which can be contacted with the bacteria described herein.

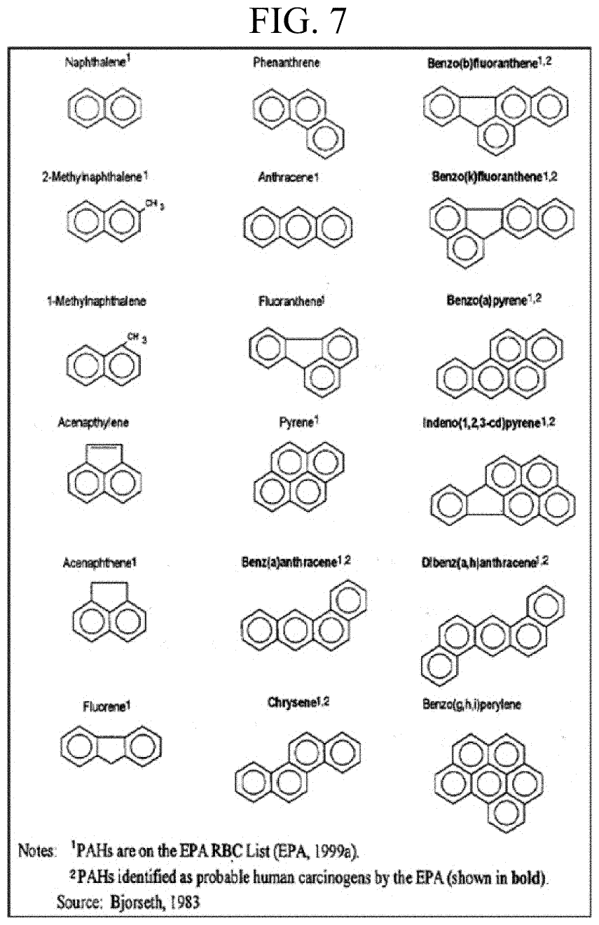

### DETAILED DESCRIPTION OF THE PREFERRED EMBODIMENTS

The exploitation of oil generates a considerable amount of produced water, which may be contaminated with petroleum products, including polycyclic aromatic hydrocarbons (PAHs) like BZP. Among these PAHs, benzo[a]pyrene (BZP) containing five fused aromatic rings is the most resistant to degradation. Contaminated water produced during oil recovery is also characterized by high salinity, e.g., up to 30% NaCl, thus the exploitation of biodegradation to remove these PAHs requires the use of active halophilic microbes.

As disclosed herein, the strain 10SBZ1A was isolated following enrichment in a mineral medium containing BZP as the sole source of carbon and containing 10 wt % NaCl. Homology analyses of 16S RNA sequences identified 10SBZ1A as belonging to the species Staphylococcus haemoliticus based on its 99.99% homology, see GenBank: MN388897.1 which describes the Staphylococcus haemolyticus strain 10SBZ1A 16S ribosomal RNA gene, partial sequence. The strain grows in the presence of 4-200 μmol of BZP as its sole source of carbon, with a doubling time of 2-10 h. Strain 10SBZ1A degrades BZP at a rate of 0.2 μmol per day which is within the range of degradation of active non-halophilic microbes that degrade BZP. Preferable, conditions for growth of this strain were a temperature of 37° C., 10% salinity and pH7. Strain 10SBZ1A is shown to actively degrade PAHs of lower molecular weights, including pyrene, phenantrene, anthracene and naphthalene; the mono-aromatic catechol and salicylic acid, and long chain fatty acids. This strain can also remove BZP in soil spiked with BZP (10 μmol in 100 mg of soil) within about 30 days. This present disclosure provides a description of a halophilic microbe that degrades BZP at a salinity of 5 wt % or more.

A method for degrading a polycyclic aromatic hydrocarbon (PAH) comprising, consisting essentially of, or consisting of contacting the PAH or a composition containing the PAH with a bacterium that is Staphylococcus haemolyticus or a bacterium having rDNA that is at least 99% identical to the rDNA of Staphylococcus haemolyticus under conditions suitable for degradation of the polyaromatic hydrocarbon is described. In some embodiments of this method the bacterium is Staphylococcus haemolyticus, preferably, the Staphylococcus haemolyticus strain 10SBZ1 A, or a modified strain derived from Staphylococcus haemolyticus strain 10SBZ1. A Staphylococcus haemolyticus strain may have genomic DNA that is at least 70, 75, 80, 85, 90, 95, 96, 97, 98, 99, 99.5, 99.9, <100 or 100% identical to that of Staphylococcus haemolyticus strain 10SBZ1A or ribosomal DNA that is at least 90, 95, 96, 97, 98, 99, 99.5, 99.9, <100 or 100% identical to that of Staphylococcus haemolyticus or which differs by no more than 1, 2, 3, 4, 5, 6, 7, 8, 9, 10 nucleotides from the rDNA of Staphylococcus haemolyticus strain 10SBZ1. BLASTN may be used to identify related polynucleotide sequences. A representative BLASTN setting modified to find highly similar sequences uses an Expect Threshold of 10 and a Wordsize of 28, max matches in query range of 0, match/mismatch scores of 1/−2, and linear gap cost. Low complexity regions may be filtered or masked. Default settings of a Standard Nucleotide BLAST are described by and incorporated by reference to hypertext transfer protocol secure://_blast.ncbi.nlm.nih.gov/Blast.cgi?PROGRAM=blastn&PAGE_TYPE=BlastSearch &LINK_LOC=blasthome (last accessed Feb. 13, 2020).

A modified, passaged, or lab-adapted strain of Staphylococcus haemolyticus, such as strain 10SBZ1, may differ from the natural isolate, for example, in rate of growth, ability to use one or more PAHs, such as those disclosed herein, as a carbon source, nutrient preference, pH preference, temperature preference, hydrophobicity, hydrophilicity, or genetically or epigenetically.

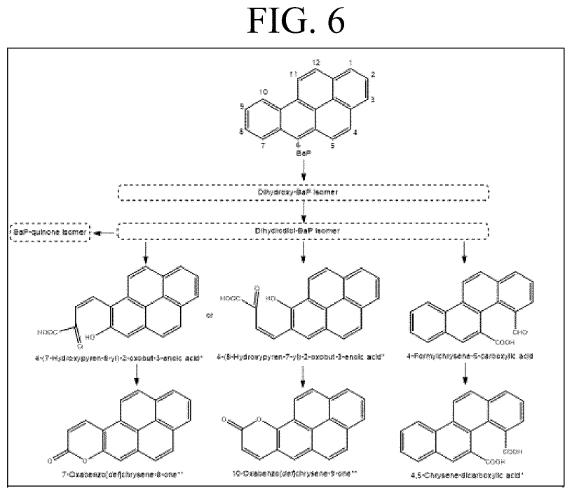

The Staphylococcus haemolyticus strains used in the method disclosed herein degrade PAHs, such as those having 2, 3, 4, 5, 6 or more rings, including those PAHs disclosed in FIG. 6. These include polycyclic aromatic hydrocarbons selected from the group consisting of naphthalene (NAPH), phenanthrene (PHEN), anthracene (ANTH), pyrene (PYR) and benzo(a)pyrene (“BZP”).

Advantageously the method of the present disclosure may be performed in aqueous saline solutions or culture media, such as those having a salt content of ≥0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or >30 wt. %. In some embodiments, a saline material containing or contaminated with a PAH may be diluted with water or an aqueous solution to contain a salt concentration within this range prior to contacting the diluted solution with Staphylococcus haemolyticus.

In other embodiments, Staphylococcus haemolyticus may be introduced into a contaminated material such as soil or a solid waste containing a PAH, onto surfaces contaminated with PAHs, such as by airborne PAHs, onto plants or crops contaminated with PAHs, or onto other biological surfaces such as skin or into mucosal or gastrointestinal tissues. In some instances, a soil will contain about 0.5, 1.0, 1.5, 2.0, 2.5, 5, 10, 15 or >15 mg of a PAH per kg and a contaminated fluid about 0.5, 1.0, 1.5, 2.0, 2.5, 5, 10, 15 or >15 mg of a PAH per liter. The level of contamination varies from one site to another or from one source of contamination to another. Nevertheless, individual PAHs are generally present in range of the 0.01-10 ppm (0.01-10 ug/L) in the environment. Any concentration of bacteria that will grow and multiply where PAHs or BZP are present may be used. In some embodiments, less than 10 bacterial cells are able to grow, multiply, and degrade a given pollutant when all other factors necessary for growth are present.

In some embodiments, contacting the bacterium with one or more PAHs occurs at a temperature ranging from 10, 15, 20, 25, 30, 31, 32, 33, 34, 35, 36, 37, 38, 39, 40, 41, 42, 43, 44, 45, or >45° C., preferably at about 36 to 38° C., at a salinity ranging from 1, 2, 5, 6, 7, 8, 9, 10, 11, 12, 13, 14, 15 to 20 wt %, and at a pH ranging from <5, 5, 6, 6.5, 6.75, 7, 7.25, 7.5, 7.75, 8, to 9.

The method may be performed in a solution or culture medium containing a long chain fatty acid such as palmitic, stearic, oleic acids, or other fatty acids having 14 or more carbon atoms, preferably 14 to 20 carbon atoms, including saturated and unsaturated fatty acids. Long chain fatty acids include the saturated fatty acids: myristic acid or tetradecanoic acid (C14:0), palmitic acid or hexadecanoic acid (C16:0), stearic acid or octadecanoic acid (C18:0), and arachidic acid or eicosanoic acid (C20:0); the monounsaturated omega-9 fatty acids: oleic acid (C18:1), eicosenoic acid (C20:1), erucic acid (C22:1) and nervonic acid (C22:1); the omega 3 polyunsaturated fatty acids: alpha-linolenic acid or octadecatrienoic acid (C18:3), stearidonic acid or moroctic acid (C18:3), eicosapentaenoic acid (EPA) or timnodonic acid (C20:5), and docosahexaenoic acid (DHA) or cervonic acid (C22:6); the omega-6 polyunsaturated fatty acids: linoleic acid (C18:2), gamma-linolenic or gamoleic acid or GLA (C18:3), dihomo-gamma-linolenic acid or DGLA (C20:3), and arachidonic acid (C20:4); and the omega-9: unsaturated acids such as mead acid (C20:3). The method may be also performed using cooking oils, glycerol esters, and/or glycerol and may be used to degrade other compounds such as mono-aromatic catechol or salicylic acid.

Another embodiment of the invention is directed to an aqueous composition in combination with a strain of a Staphylococcus haemolyticus strain that degrades or which uses one or more PAHs as a carbon source. Preferably, the composition will contain a strain that grows in the presence of 1, 2, 5, 10, 15 or 20 wt. % NaCl and which can utilize BZP or another PAH as its sole carbon source. The composition may contain a suitable growth medium for Staphylococcus haemolyticus such as a medium containing PAH or PAHs as sole carbon sources, a medium containing other carbon sources, nitrogen sources, salts, minerals or vitamins or buffers, Bushnell-Hass (BH) medium, or a richer medium such as Luria-Bertani Broth (LB) culture medium.

Materials containing nitrogen, phosphorus and essential minerals, trace elements, and vitamins may be used to feed microbes during the degradation of PAHs. One or more ingredients present in a conventional Staphylococcus haemolyticus growth or culture media may be added. For example, one or more complex hydrocarbons besides the PAH to be degraded, including crude oil, diesel fuel and lubricating oil may be included or simple hydrocarbons such as octane, decane, hexadecane, methylcyclopentane, methylcyclohexane, heptamethylnonane, benzene, toluene, ethylbenzene, m-, o- and p-xylenes, naphthalene, carbazole, anthracene and phenanthrene, may be used as additional carbon sources. One or more components of a basal medium may be included such as KH₂PO₄, NH₄Cl, MgCl₂ CaCl₂, yeast extract or trace element solution. Preferably for sustained growth to take place nitrogen, phosphorous, and other essential nutrients may be provided.

In some embodiments, a contaminated fluid or solid, such as soil, is contacted with the Staphylococcus haemoliticus while the fluid is exposed to oxygen (e.g. air, oxygen-containing gas mixture) or light (e.g., sunlight, artificial illumination) at a pressure of about 0.1-10 bar, preferably about 1 bar. 02 may be from atmospheric air and/or other sources, such as an 02 gas tank, from which 02 may be pumped or injected into a contaminated fluid or solid before and/or while the fluid or solid. Water may be present in the contaminated fluid or may be introduced into the fluid in the form of liquid water or water vapor.

In another embodiment, Staphylococcus haemolyticus, such as an attenuated or non-infectious variant of Staphylococcus haemolyticus may be applied to the skin or mucous membrane, or ingested or incorporated into the gastrointestinal tract or respiratory system to degrade PAHs that have been deposited, ingested or inhaled by a human or non-human animal.

In another embodiment, Staphylococcus haemolyticus, may be sprayed or otherwise applied to surfaces, including plant surfaces such as leaves, stalks, flowers or fruits, that have been contaminated with a PAH, such as with an airborne PAH.

In one embodiment the aqueous composition comprises, consists essentially of, or consists of an aqueous medium containing a polycyclic aromatic hydrocarbon, and at least 5, 10, 15 or 20 wt. % NaCl, and containing a Staphylococcus haemolyticus strain that can utilize BZP or another PAH as its sole carbon source. Any medium suitable for growth or viability of Staphylococcus haemolyticus may be used. One or more PAHs may be present in or introduced into the medium. In some embodiments, the Staphylococcus haemolyticus may be attached to a bead, film or other solid substrate around which, or over which, medium is passed.

Another embodiment of the invention is directed to a Staphylococcus haemolyticus strain that has been genetically or epigenetically modified to increase its growth rate in a medium containing NaCl and/or increase its growth rate in the presence of a PAH. Such a strain may be derived from strain 10SBZ1A by genetic or epigenetic modification to increase its growth rate or the rate at which it degrades a PAH compared to a parent strain 10SBZ1A in a medium containing at least 5 wt % NaCl or in the presence of BZP or other PAHs.

Modifications may be introduced by procedures known in the art including by serial passage and selection, exposure to mutagens such as UV light, ethyl methane sulphonate (EMS) and 1-methyl 3-nitro 1-nitrosoguanidine (NTG). Mutagenesis procedures are incorporated by reference to Kamath, et al., Bioresource Technology, Volume 99, Issue 18, December 2008, Pages 8667-8673.

A genetically modified Staphylococcus haemolyticus may have one or more genetic modifications including those that induce auxotrophy for one or more molecules made by the unmodified strain, addition or deletion of antibiotic resistance, deletion or including of one or more polynucleotides encoding a toxin, or the incorporation of expression control sequences such as inducible or repressible promoters into the genome or episomes of the bacterium. In some embodiments, one or more toxins, such as SEA, SEB, SEC, SED, SEE or one or more enterotoxins may be absent or deleted from a S. haemolyticus strain for example, by deletion or inactivation of genomic or episomal DNA encoding such toxins.

A modified Staphylococcus haemolyticus variant may have one or more epigenetic changes to its DNA, such as a variant methylation or hydroxymethylation pattern in its genomic DNA, a difference in histone methylation, or difference in microRNA expression, compared to an otherwise identical natural isolate. Epigenetic variants are those having a heritable phenotype change that does not involve alterations in its DNA sequence. In one embodiment, a laboratory strain degrades BZP more efficiently, at a greater rate or to a greater extent (lower resulting concentration) at a higher salinity, such as up to 5, 6, 7, 8, 9, 10 or >10% NaCl (w/v) than the corresponding natural isolate.

Contamination levels. The proportion of the PAH or other contaminants present in a contaminated fluid or solid may vary, for example, depending on the source of the contaminated fluid or solid and for aqueous environments on the hydrophobicity of the PAH. For example, PAHs are often present in soil and river sediments near industrial sites such as creosote manufacturing facilities and can be distributed by oil spills, use of creosote, coal mining dues and other fossil fuel sources. In some embodiments, the concentration of the PAHs, such as BZT, in a contaminated liquid or solid ranges from 0.01 to 20 ppb, or from 0.02 to 10 ppb, or from 0.2 to 1 ppb. For air or gaseous materials, a PAH content can range from 0.01 to 20, 0.02 to 10 or 0.2 to 1 mg/m³.

In situ processing. The degradation of PAHs may involve holding pools/ponds, effluent catches, or other artificial or natural reservoirs or bodies of water containing produced water (PW) or other wastes contaminated with BZP or other PAHs or dedicated to bioremediation of PAHs. Landfills and other solid waste dumps or disposal areas may also be used as a source of BZP carbon. These may be inoculated or otherwise contacted with a microbe according to the invention under conditions analogous to those described below for a bioreactor.

In some embodiments, the Staphylococcus haemolyticus may be pumped into contaminated ground water. In some embodiments, air is pumped into a body of contaminated PW or other liquid, or periodic or intermittent agitation may be applied to facilitate contact between microbes and contaminants. In some in situ methods, pellets or particles containing viable halophilic microbes are introduced. These may sediment or embed in contaminated sludge at the bottom of the pool, reservoir or other body of water and release viable halophilic bacteria, or halophilic bacteria and nutrients, over a prolonged period of time, such as over a period of one day, week, one month, three months, or one year or more (or any intermediate time period).

Halophilic Staphylococcus haemolyticus may be encapsulated in a polymer or matrix of choice which may be designed to slowly dissolve and release viable halophilic bacteria at different temperatures, pHs, or at different salt concentrations such as any of those described elsewhere herein. In some embodiments the bacteria can be encapsulated in, or incorporated into or on, a granule, a pellet, a powder, a woven fabric, a non-woven fabric, a mat, a felt, a block, a honeycomb or other substrate such as a flowable surface.

A mixture of different polymers containing Staphylococcus haemolyticus or other halophilic microbes may be applied, for example, as separate mixtures for each of the microbial strains and may be prepared and designed to release viable microbes at optimal or preferred pH, saline concentrations, or temperatures. Alternatively, viable halophilic Staphylococcus haemolyticus as disclosed herein may be introduced on lighter materials that float on or become suspended in a contaminated pool, reservoir or body of water to increase the surface area of exposure between the microbes and contaminated water or other material.

Bioreactor. In some embodiments, the method of the invention is performed in part or in whole in a bioreactor. These may include “pump and treat” methods in which contaminated groundwater is pumped to the surface, cleaned by passing the groundwater through a bioreactor, and then reinjected into the groundwater. The bioreactor may be one element in a system of mechanical or electronic elements that process and control the flow and exposure of a material containing a PAH to a microorganism of the invention. The method may also be performed in two or more bioreactors which may be arranged sequentially and independent supplied with nutrients that facilitate degradation of PAHs. In other embodiments, the method of the invention may be performed directly in a liquid solution that contains PAHs without a bioreactor by the inoculation of the solution with one or more microorganisms disclosed herein and/or nutrients.

Material, such as a liquid or flowable material such as produced water (“PW”), contaminated with polycyclic aromatic hydrocarbons (PAHs) may be directed into a bioreactor or system containing one or more bioreactors, and inoculated or otherwise contacted with the microorganisms described herein, so that the PAHs are subsequently degraded. Such system may assay PAH levels and recirculate a contaminated material until a desired low level of a PAH contaminant is attained.

In some embodiments, a PAH, such as an airborne or circulating PAH, may be incorporated into a fluid medium, which optionally contains a surfactant to increase the solubility of the PAH in an aqueous medium prior to exposure of the aqueous medium to the bacteria. Surfactants include anionic, nonionic and cationic surfactants such as those described by hypertext transfer protocol secure://en.wikipedia.org/wiki/Surfactant in biologically compatible or nontoxic concentrations. Usable biosurfactants include any of those described by hypertext transfer protocol://www.biotechnologynotes.com/industrial-biotechnology/biosurfactants/biosurfactants-definition-classification-production-and-applications-industrial-biotechnology/14024 (last accessed Feb. 21, 2020).

The bioreactor may be configured to maintain a suitable salinity (e.g., 5, 10, 15, 20, 25, 30 wt % NaCl, preferably between 5 and 15 wt %), a suitable pH preferably between 6.5 and 7.5, a suitable temperature, such as one at or above 25, 30, 40, 50, 55, or 60° C., preferably about 37° C., as well as adequate levels of oxygen, nutrients, alternative carbon sources, sugars, vitamins, salts, minerals, trace elements, a nutrient medium, antioxidants, carriers, buffers, or other excipients or cofactors conducive to degradation of pyrene or other PAHs.

A bioreactor can include other materials besides the Staphylococcus haemolyticus including other microorganisms such as bacteria or fungi, or adsorbents such as activated carbon, graphite, activated alumina, a molecular sieve, aluminophosphate material, silicoaluminophosphate material, zeolites, faujasite, clinoptilolite, mordenite, metal-exchanged silicoaluminophosphate, uni-polar resin, bi-polar resin, aromatic cross-linked polystyrenic matrix, brominated aromatic matrix, acrylic polymer, acrylic copolymer, methacrylic polymer, methacrylic copolymer, hydroxyalkyl acrylate, hydroxyalkyl methacrylate, adsorbent carbonaceous material, adsorbent graphitic material, carbon fiber material, nano-material, adsorbent metal salts (including, but not limited to perchlorates, oxalates, and alkaline earth metals), alkaline earth metal metallic particles, ion exchange resin, linear polymers of glucose, polyacrylamide, or a combination thereof, to advantageously adsorb from the fluid PAHs or other inorganic or organic contaminants such as benzene, xylene, toluene, phenol, ethyl benzene, heavy metal ions, or dyes; or dead or degraded microorganisms.

Batch processing. In batch like systems, the contaminated PW or other material may be held statically, or periodically agitated or mixed, until a desired lower level of PAH contaminant is attained.

Soil or biopile. The method of the invention may be performed in a biopile, soil or other solid or semisolid material by inoculation or incorporation of the microorganisms into a material containing a PAH. The method may be conducted under similar conditions (pH, temperature, nutrient addition, etc.) as described for the method as performed in a bioreactor. Biopiles, also known as biocells, bioheaps, biomounds and compost piles, are used to reduce concentrations of polycyclic aromatic hydrocarbons including petroleum constituents in soils through the use of bioremediation. Biopiles are above ground, engineered systems that use oxygen, generally from air, to stimulate the growth and reproduction of aerobic bacteria which, in turn, degrade the petroleum constituents adsorbed in soil. Biopiles are commonly aerated by forcing air to move through the biopile by injection or extraction through slotted or perforated piping placed throughout the pile. In some embodiments, the invention involves heaping contaminated soils into piles and stimulating microbial activity in the soils through addition of a microbe according to the invention, other hydrocarbon degrading microbes, aeration and/or nutrients and moisture.

Carriers. The microbes disclosed herein may be introduced in free form or introduced on a carrier. For example, a single or multiple types of halophilic bacterial described herein may be adsorbed on suitable carrier and dispersed in soil while being supported on a carrier. The carrier used includes any known material so far as it can be applied to or incorporated into a contaminated soil or other solid or semisolid material. A carrier may be admixed, adsorbed to, or infused with one or more microbes of the invention, other microbes, nutrients, or PAH-containing material to be remediated or degraded by microbial action. A carbonaceous carrier such as carbon black or charcoal is preferred. A carrier may act as a growth substrate and facilitate multiplication and spreading of microorganisms, such as Staphylococcus haemolyticus, or increase its contact with a PAH in a contaminated material. It may act as a nutrient source, including as a gradual or time-released nutrient source, for the microorganisms thus applied, particularly a nutrient source, which can be gradually released to advantage. Preferably, a carrier will be biodegradable. Further, the carrier is a biodegradable material so that after action of a microorganism of the invention it may be removed leaving the treated material or site in a more natural state. A carrier or bulking agent may be added at a ratio of 1-<100% wt/wt % of the hydrocarbon waste-soil mixture, for example, from 5-50 wt % or from 10-30 wt %, or any intermediate value or subrange. A carrier or bulking agent may comprise, agricultural wastes, tree litter including fronds, leaves, needles, wood chips, bark, grain or maize husks or cobs, bagasse, bark, post peelings, fruit waste, stones, individually or in mixture thereof. These may be ground or granulated to increase surface area and to facilitate mixture into a solid or semisolid material.

A carrier may contain water, for example, it may contain about 0.1% to 99% by weight, 5% to 90% or 10% to 85% by weight of water or another liquid non-toxic to microorganisms used to degrade PAHs. It may contain one or more nutrients for the microorganisms according to the invention. When water content of the carrier is too low, microorganisms do not thrive and struggle to replicate and survive. In contrast, when the water content of the carrier is too high, the resulting carrier exhibits a deteriorated physical strength that makes itself difficult to handle. The carrier adsorbed microbial blend is tested for its efficacy in liquid medium as well in a biopile.

According one embodiment of the invention, the biopile comprises a layer of soil at the base, a layer of the mixture of the aromatic waste, soil and a bulking agent, a pipe for aeration, drainage and addition of water. The pipe may a perforated polyvinyl chloride (PVC) pipe wrapped with fine cloth, a layer of soil above the mixture and a layer of perforated plastic sheet to cover the biopile and avoid volatile organic compounds (VOCs). The water content within the biopile is maintained by sprinkling the water at the top and sides of the biopile or by using water reservoir or irrigation system (e.g., drip or soaker hoses). The addition of pH adjuster (e.g., to provide an alkaline pH), nutrients and moisture may be combined. The moisture content of the biopile is maintained preferably from 30% to 80% of the water retention capability of the soil-aromatic hydrocarbon waste mixture. When the water content is too low, microorganisms find difficulty in survival. On the contrary, when the water content is too high, it stops aeration. The aeration is maintained by passive diffusion of air using perforated pipes, extraction or injection of air with suitable device or by tilling at every month interval.

Biosensors. In some embodiments, the microbes of the invention may be employed as biosensors to identify and quantify target compounds such as PAHs like BZP or particular PAHs through interaction of the microbes with these compounds. Growth rate may be used as one indicator of the presence of absence of a PAH or, alternatively, genetic markers including quantity of mRNA expression or expression of reporter genes, involved with degradation of a PAH. For example, the Staphylococcus haemolyticus disclosed herein may be modified to indicate the presence of a PAH or the expression of a microbial gene used to degrade a PAH. Methods of introducing fluorescent and other reporter genes (GFP, lux, luc) into a microbe and evaluating a change in metabolic status are known in the art, for example, as described by Troegl, et al., Sensors (Basel) 12(2):1544-157 (2012) which is incorporated by reference.

The method of the invention may be used in an industrial, commercial, domestic or other environment for degradation or remediation of compositions contaminated with PAHs and associated compounds. For example, the method of the present disclosure can be practiced wherever undesired PAHs are produced or deposited including those produced by the oil or petroleum industry, plastics industry, agriculture, food or drink industry, clothing industry, packaging industry, electronics industry, computer industry, environmental industry, chemical industry, aerospace industry, automotive industry, biotechnology industry, medical industry, healthcare industry, dentistry industry, energy industry, consumer products industry, pharmaceutical industry, mining industry, cleaning industry, forestry industry, fishing industry, leisure industry, recycling industry, cosmetics industry, pulp or paper industry, textile industry, clothing industry, leather or suede or animal hide industry, tobacco industry or steel industry.

### EXAMPLES

Material and Methods. The chemical compounds (NH₄)₂SO₄, KH₂PO₄, CaCl₂.7H₂O, MgSO₄.7H₂O, Na₂HPO₄ and FeSO₄.7H₂O, BZP, PYR, ANT, PHEN, NAPH, phthalic acid, salicylic acid (SALC), catechol (CATC), were purchased from Sigma-Aldrich (St. Louis, Mo., USA). Chemical for the Luria-Bertani Broth (LB) culture medium were obtained from Difco (Lawrence, Kans., USA).

Microbial isolation. Soil samples, collected from an oil-contaminated area in the coastal line of the Arabian Gulf, were used as inoculum for the isolation of BZP degrading microbes. This isolation was carried out by enrichment cultures, initiated with 1.0 g of soil samples in 50 ml of medium. This medium, known as Bushnell-Hass (BH), consisted of (NH₄)₂SO₄ (2.38 g), KH₂PO₄ (1.36 g), CaCl₂.7H₂O (10.69 g), MgSO₄.7H₂O (0.25 g), Na₂HPO₄ (1.42 g) and FeSO₄.7H₂O (0.28 mg) per liter, and supplemented with 0.05% (wt/v) [0.5 g⁻¹] of BZP as a sole carbon source, and this medium was referred as BH—BZP. Bacteria were cultured at 37° C., at 120 rpm for 3-4 weeks, and thereafter, were transferred to a fresh BH—BZP medium ( 1/10, v/v) for another 2-3 weeks. The process was repeated 4-5 times until bacterial growth was observed.

To isolate individual bacterial strains, the culture was streaked on (1%, m/v) agar plate dissolved in BH—BZP, and then incubated at 37° C. for 15-21 days. The resulting individual colonies were streaked again in new plates to ascertain their purity, and then cryopreserved in 15% glycerol for further studies.

Scanning electron microscopy (SEM) analysis. SEM analysis was carried out as reported elsewhere; see Nzila, A, A Thukair, et al. (2016). Isolation and characterization of naphthalene biodegrading Methylobacterium radiotolerans bacterium from the eastern coastline of the Kingdom of Saudi Arabia. Arch Environ Prot 42(3): 25, Nzila et al. (2017), each incorporated herein by reference in their entirety.

Briefly, bacteria were immobilized on microscope cover-slides and fixed in formaldehyde (5%, v/v) for 12 h, followed by dehydration in a series of ethanol-water solutions (ethanol 30%, 50%, 70%, 80%, 90% and 95%, v/v). The samples were then sputter-coated with gold (Eiko, Japan) prior to observation under SEM (JSM-T300, JEOL, Japan).

Species identification. Bacterial species were identified with 16S rRNA sequencing and alignment as previously reported and incorporated by reference to Nzila, A, et al. (2016b). Isolation and characterization of naphthalene biodegrading Methylobacterium radiotolerans bacterium from the eastern coastline of the Kingdom of Saudi Arabia. Arch Environ Prot 42(3): 25-32.

Briefly, after bacterial growth and lysis, DNA was purified with InstaGene Matrix for PCR analysis. Primers 1492F AGAGTTTGATCMTGGCTCAG (SEQ ID NO: 1) and 1492R TACGG YTACCTTGTTACGACTT (SEQ ID NO: 2) were used, resulting in a 1400 bp (base pairs) PCR product. Thereafter, this PCR product was sequenced by using Big Dye terminator cycle sequencing kit (Applied BioSystems, USA) with the following primers 518F: CCAGCAGCCGCGGTAATACG (SEQ ID NO: 3) and 800R: TACCAGGGTATCTAATCC (SEQ ID NO: 4) and sequenced in an automated DNA sequencing system (Applied BioSystems model 3730XL, USA). Species identification was carried out by Basic Local Alignment Search Tool (BLAST), available at the National Center for Biotechnology Information Database (NCBI).

Bacterial growth assessment in the presence of BZP and various hydrocarbon molecules: effect of pH and salinity. To initiate these experiments, bacteria were pre-cultured in an LB rich medium, and around 5×105 colony forming units (CFU) of these bacteria were added to 50 ml BH medium containing 100 μmol BZP, unless otherwise stated. Non-polar substrates pyrene, phenanthrene, naphthalene and anthracene were dissolved in dimethyl sulfoxide (DMSO) [0.1 g/ml], while the water-soluble substrates SALC and CATC were dissolved directly in BH medium. The aliphatic compounds were directly added to the medium. Bacterial growth in the presence of BZP was also assessed at temperatures between 30 35, 40 and 45° C.; salinity at 0, 5, 10, 15 and 20% of NaCl [wt/wt]), and pH 5, 6, 7, 8 and 9. Growth was monitored as a measure of CFU/ml.

Bacterial doubling time (dt) was calculated by fitting growth curves data points into Qₜ=Qₒe^{-kt} equation, where Q is the bacterial CFU ml⁻¹ at time t, Qₒ is the initial bacterial count in the culture, and k is the growth rate. The growth rate (k) was then used to compute dt values according to the equation: dt=ln(2)/k (in days). Values were calculated with Microsoft Excel software (Version). All experiments were carried out in duplicate.

Quantification of BZP and substrate utilization. The remaining BZP was quantified in a bacterial culture of 100 ml containing 20 μmol BZP for 18 days, according to the protocol reported by and incorporated by reference to Budiyanto, F, et al. (2017). Characterization of halophilic bacteria capable of efficiently biodegrading the high molecular weight polycyclic aromatic hydrocarbon pyrene. Environmental Engineering Science. June 2018.616-626.

A sample corresponding to one culture flask of 100 ml, was collected every 5 days and subjected to extraction as follows. The samples were sonicated for 30 min and then extracted twice with ethyl acetate (50 ml). The combined organic layers were dehydrated with calcium chloride and then dried under vacuum. Samples were re-dissolved in 500 μl of chloroform prior to analysis by gas chromatography-mass spectrometry (GC-MS). Concentrations of BZP in the samples were obtained with a 5-point standard curve (4, 20, 100, 200, 400 μmol 1⁻¹) of BZP in BH medium. Values of quantified BZP were fitted in the exponential decay equation, Qₜ=Qₒe^{-kt} to obtain the biodegradation rate constant (k); see Lu, J, C Guo, et al. (2014). Biodegradation of single pyrene and mixtures of pyrene by a fusant bacterial strain F14. International Biodeterioration & Biodegradation 87: 75-80, incorporated herein by reference in its entirety.

Quantification of BZP utilization in soil samples. The ability of the strain to degrade BZP in the context of soil contamination was also evaluated. Around 100 mg of soil sample (consisting primarily of sand) was spiked with 1 μmol.1⁻¹ of BZP, and place in a 5 cm×5 cm Petri dish. An approximate amount of 10⁷ CFU was added to this 100 mg soil, along with BH medium to keep the soil wet. Control experiments were prepared without adding bacteria. At day 0, 15 and 30, each sample (from 5 cm×5 cm Petri dish) were sonicated and extracted as previously explained and subjected to GC analysis for quantification.

Detection of metabolites by GC-MS. Bacteria were grown for 15 days in 1.0 μliter of media containing BZP at 4000 μmol.1⁻¹. The medium was then filtered, and subjected to NaOH/HCL extraction to remove the non-polar compounds, which are primarily the un-utilized BZP. Thereafter, the medium, containing metabolites, which are primarily polar compounds, was subjected to extraction with ethyl acetate (100 ml×3), as explained above.

The resulting dried pellet was dissolved in 1.0 ml of chloroform and divided into two portions. One portion was analyzed by GC-MS and the other portion was evaporated, dried and then subjected to derivatization with trimethylchlorosilane. This process, also known as silylation, consisted of mixing the remaining residue with pyridine (40 μl), N,O-bis-trimethylsilyl acetamide (40 μl) and trimethylchlorosilane (20 μl), followed by an incubation at 80° C. for 10 min under a nitrogen atmospheric condition; see Berthet, A, et al. (2011). Gas-chromatography mass—spectrometry determination of phthalic acid in human urine as a biomarker of folpet exposure. Anal Bioanal Chem 400(2): 493-502; and Hadibarata, T., et al. (2013). Biodegradation and metabolite transformation of pyrene by basidiomycetes fungal isolate Armillaria sp. F022. Bioprocess Biosyst Eng 36(4): 461-468, each incorporated herein by reference in their entirety.

The mixture was then diluted with 1.0 ml of chloroform prior to its analysis by GC-MS. Metabolites were identified with an Agilent 6890N GC equipped with an HP-5 [30 m, 0.25 mm 191 (i.d.)] column using helium as the carrier gas attached to a 5975B MS. The conditions of the ethyl acetate (50 ml). The combined organic layers were dehydrated with calcium chloride and then dried under vacuum. Samples were re-dissolved in 500 μl of chloroform prior to analysis by gas chromatography-mass spectrometry (GC-MS). Concentrations of BZP in the samples were obtained with a 5-point standard curve (1, 5, 25, 50, 100 mg⁻¹) of BZP in BH medium. Values of quantified BZP were fitted in the exponential decay equation, Qₜ=Qₒe^{-kt} to obtain the biodegradation rate constant (k); see Lu et al., International Biodeterioration & Biodegradation 87: 75-80.

Quantification of BZP utilization in soil samples. The ability of the strain to degrade BZP in the context of soil contamination was also evaluated. Around 100 mg of soil sample (consisting primarily of sand) was spiked with 2.5 mg of BZP, and place in a 5 cm×5 cm Petri dish. An approximate amount of 10⁷ CFU was added to this 100 mg soil, along with BH medium to keep the soil wet. Control experiments were prepared without adding bacteria. At day 0, 15 and 30, each sample (from 5 cm×5 cm Petri dish) were sonicated and extracted as explained above, and subjected to GC analysis for quantification.

Detection of metabolites by GC-MS. Bacteria were grown for 15 days in 1.0 μliter of media containing BZP at 1000 mg 1⁻¹. The medium was then filtered, and subjected to NaOH/HCL extraction to remove the non-polar compounds, which are primarily the un-utilized BZP. Thereafter, the medium, containing metabolites, which are primarily polar compounds, was subjected to extraction with ethyl acetate (100 ml×3), as explained in previous paragraph. The resulting dried pellet was dissolved in 1.0 ml of chloroform and divided into two portions. One portion was analyzed by GC-MS and the other portion was evaporated, dried and then subjected to derivatization with trimethylchlorosilane. This process, also known as silylation, consisted of mixing the remaining residue with pyridine (40 μl), N,O-bis-trimethylsilyl acetamide (40 μl) and trimethylchlorosilane (20 μl), followed by an incubation at 80° C. for 10 min under a nitrogen atmospheric condition; see Berthet et al., Anal Bioanal Chem 400(2): 493-502 and Hadibarata, et al. Bioprocess Biosyst Eng 36(4): 461-468. The mixture was then diluted with 1.0 ml of chloroform prior to its analysis by GC-MS.

Metabolites were identified with an Agilent 6890N GC equipped with an HP-5 [30 m, 0.25 mm (i.d.)] column using helium as the carrier gas attached to a 5975B MS. The conditions of the analysis were: initial temperature of 50° C. held for 2 min followed by an increase to 250° C. at a rate of 5° C./min and holding time at 250° C. for 30 min. See Budiyanto et al. The MS analysis conditions were: inlet temperature at 250° C. and mass range of 15-550 m/z.

Statistical analyses. Statistical analyses were carried out using one-way analysis of variance (ANOVA), t-test and a simple linear regression fitting model, and the strength of linearity was assessed based on the Pearson correlation coefficient. In all tests, p<0.05 was considered to be the level of significance. The software MINITAB (Version 16, Coventry, UK) was employed in these analyses and is hereby incorporated by reference.

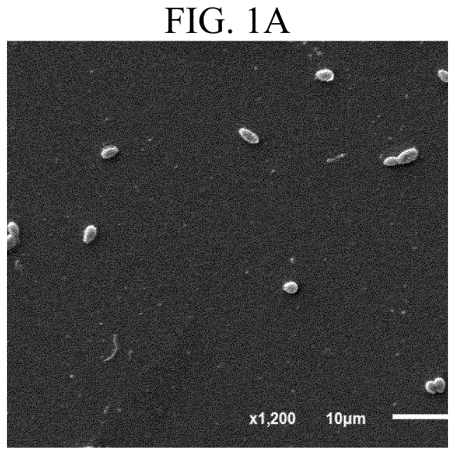

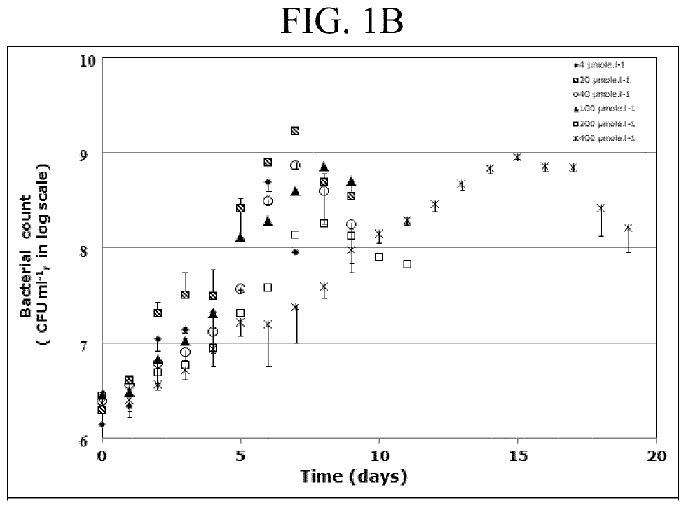

Enrichment, strain isolation and species identification. The enrichment protocol led to the isolation of one strain, 10SBZ1A, capable of growing in the presence of BZP as sole substrate. Under light microscope, 10SBZ1A colonies were white/cream, with circular form, flat and entire margin. The bacteria were Gram-negative and electron microscopy showed that they had a crocus-shape and diameters of about 1.2 μM (FIG. 1A).

DNA was purified from strain 10SBZ1A and subjected to 16S rRNA gene sequencing for species identification. Using the Basic Local Alignment Search Tool (BLAST) program for homology analysis of available 16S rRNA gene sequences in the National Center of Biotechnology Institute (NCBI) database, this strain 10SBZ1A was identified as Staphylococcus haemoliticus, based on the threshold of 99% homology (NCBI accession number GI: MN388897). The inventors show herein that this strain can degrade the highly complex PAH BZP.

Effect of increasing of BZP concentration. Several reports have documented the toxicity of PAHs against bacteria and that high concentrations are inhibitory to bacterial growth. Consequently, the effects of 4, 20, 100, 200, and 400 μmol.1⁻¹ BZP were assessed on the bacterial growth of strain 10SBZ1A.

At BZP concentrations ranging from 5 to 20 μmol.1⁻¹, the strain showed a rapid growth, reaching the maximum growth within 6-8 days, with the maximum count falling between 10⁷-10⁸ CFU/ml, except that at 20 μmol.1⁻¹, with a this maximum count >10⁹ CFU ml⁻¹, see FIG. 1B. However at a BZP concentration of 400 μmol.1⁻¹ the bacterial growth rate decreased, as shown by the time delay at which the maximum growth was achieved, which was at day 15.

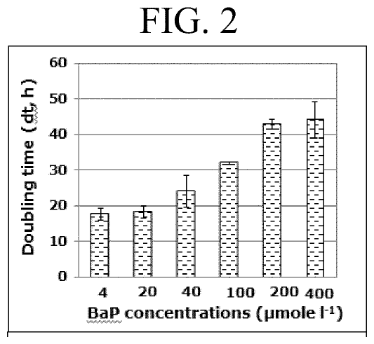

This BZP inhibitory effect was also confirmed by the increase in the culture doubling time (dt) as the BZP concentration increases as shown by FIG. 2. At concentrations ranging from 1 to 100 μmol.1¹, dt values were between 15-20 h, and these values almost doubled at 200 and 400 μmol.1⁻¹ concentrations.

The single regression ANOVA showed the correlation between dt values and BZP concentrations is statistically significant (p<0.05). This correlation followed a linear equation dt=0.0038 C+0.503 (R²=0.98, where C is pyrene concentration).

The inventors evaluated pyrene concentrations between 1 to 400 μmol.1⁻¹ and the results indicated that the increase in BZP concentration was associated with decrease in bacterial growth.

Effect of pH. The ability of strain 10SBZ1A to degrade BZP was tested at pH 5, 6, 7, 8, and 9. At pH 7 dt values were 21.49±0.98, however at other pH values the growth was slow. Thus, a preferable pH for growth of this strain and degradation of BZP was determined to be between pH 6.5 to 7.5, most preferably about pH 7.

Temperature. The effect of temperature on BZP degradation was evaluated at 35, 37, 40 and 45° C. (Table 1).

TABLE 1
Doubling time (dt, in hours) of the degradation of benzo(a)pyrene
(BaP) by Staphylococcus haemoliticus 10SBZ1A strain,
as a function of pH, temperature and salinity)

| | Conditions | Doubling time (dt, h) |
|---|---|---|
| pH | 5 | ND^{a} |
| | 6 | ND |
| | 7 | 21.5 ± 1.0 |
| | 8 | ND |
| | 9 | ND |
| Temperature | 35° C. | 28.5 ± 2.4^{b} |
| | 37° C. | 21.5 ± 1.0^{b} |
| | 40° C. | 24.5 ± 1.0 |
| | 45° C. | ND |
| Salinity (% NaCl) | 0 | ND |
| | 5% | 46.8 ± 7.4 |
| | 10% | 21.5 ± 1.0 |
| | 15% | 156 ± 74 |
| | 20% | ND |

^{a}Not determined due to slow growth rates.
^{b}The difference of dt values were statistically significant (p < 0.05).

Overall, the range of dt fell between 21-29 h, and the temperature 37° C. corresponded to the smallest dt (21+1 h). Thus, the strain 10SBZ1A was more active in degrading BZP at 37° C., although these dt differences were not statistically significant (p>0.05).

Salinity. Strain 10SBZ1A was isolated in medium containing 10% NaCl. To establish it salinity range, its growth was assessed at 0, 5, 15 and 20% NaCl, and the result compared with that obtained at 10% NaCl. No growth was observed at 0 and 20% NaCl. The higher growth rate, as measured by dt values (21±1 h) was observed at 10% NaCl, followed by a dt of 44±7 h at 5% NaCl and 156±73 h at 15%. These differences were statistically significant p<0.05, and suggested that the optimum salinity of S. haemolyticus is about 10% NaCl.

The isolated 10SBZ1A strain also grew at a similar NaCl concentration when cultured in the presence of BZP as sole source of carbon. Based on these data, the isolated strain 10SBZ1A is preferably grown in the presences of PAHs such as BZP at a salinity between 5 and 15 wt. % NaCl, more preferably at about 10 wt. % NaCl.

Utilization of other PAHs. The ability of 10SBZ1A strain to degrade the lower PAHs PYR, PHEN, ANT and NAPH was assessed. The mono-aromatic compound SALC and CATC were also included because they were proven to be part of intermediates of PAHs degradation. In the scale of degradability, aliphatic compounds are easier to degrade than aromatic ones, and microorganisms that degrade aromatic compounds can actively utilize aliphatic ones. The growth profile of 10SBZ1A strain in the presence of the long chain acids such as palmitic, stearic, oleic acids, cooking oil, which consists of ester glycerol and of long chain fatty acids of glycerol, and glycerol were also tested.

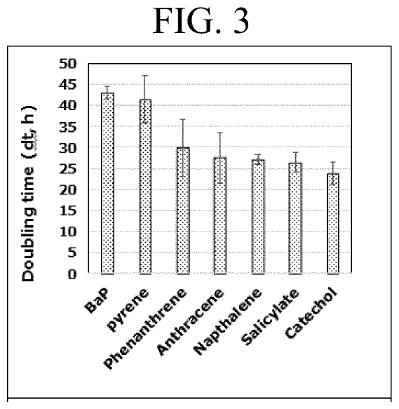

As shown in FIG. 3, overall, the ability of 10SBZ1A to degrade PAHs increased as the PAH ring number decreased. The strain dt in the presence of BZP was around 42 h, and although it remains similar with pyrene (5-ring compounds), however this value decreased to 30-33 h for phenanthrene and anthracene (3 rings), and naphathalene (2 rings).

The lower dt values were associated with the monoaromatic SALC and CATC (23-26 hours); see FIG. 3. The relationship between the number of rings and dt values followed as: dt=18.77+4.636×N (p<0.001, R2=66%) where N stands for the number of rings.

The ANOVA test for simple regression showed a strong linear correlation between dt values and the substrate's number of aromatic rings, based on the following equation: dt=−0.1253+0.3924×N (p=0.001, R2=77.1%) where N stands for the number of rings. This is in line with reports indicating the ability of some BZP-degrading bacteria to utilize PAHs of lower molecular weight.

The data also showed the higher ability of the 10SBZ1A strain to degrade long chain aliphatic acids compared to aromatic. Indeed, the strain dt fall within 15-19 hours for the 3 tested fatty acids. The 3 carbon molecule, glycerol, was associated with the highest growth profile, with a dt of around 11 hours. Interestingly, cooking oil, which consists of fatty acid and glycerol supported more growth than the fatty acids. As said earlier, cooking oil is hydrolyzed to long chain fatty acids and glycerol, thus, it is likely that the availability of this glycerol that accounts for a high growth profile associated with cooking-oil compared to long chain fatty acids. Based on these data the 10SBZ1A strain can be used for the removal of both aliphatic and aromatic pollutants.

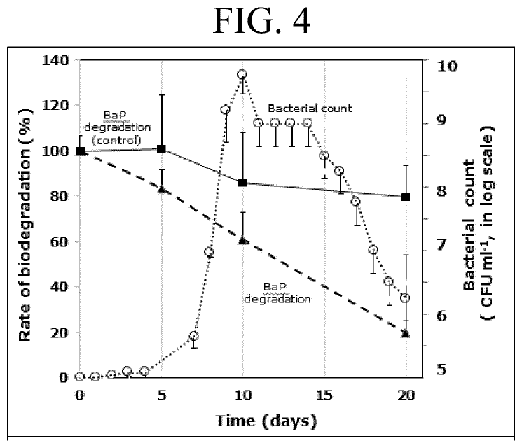

Quantification. The degradation profile shows that this strain degrades 50% of 5 mg/l BZP at day 12.5, and at day 25, almost 80% BZP was removed, leading to a BZP rate of degradation of 0.8 μmol/L/day (FIG. 4). Around 20% of abiotic degradation was observed as indicated by the control. This degradation rate was more pronounced when bacterial growth entered exponential phase.

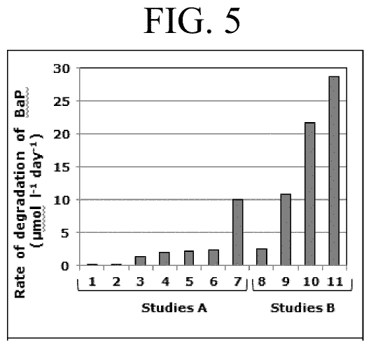

The rate of BZP degradation in 10SBZ1A was compared to those that have been reported in similar studies which were all carried out using non-halophilic microbes and these results are summarized in FIG. 5. Studies 1-7 shown in FIG. 5 involved single bacterial strains which were cultured in minimum mineral media containing BZP as the sole source of carbon. BZP degradation rates fell between 0.04 to 3p mol/L/day. The inventors found that under similar experimental conditions, the halophilic 10SBZ1A strain had a BZP degradation rate in the range of those of non-halophilic bacteria, highlighting its potential in bioremediation.

BaP Metabolite identification. In an effort to identify the BaP metabolic pathway using 10SBZ1A strain, reversed-phase HPLC was used to separate metabolites along with TOFMS/MS to analyse BaP metabolites. One of the identified metabolites, at retention time of 22.92 min, had [M+1]⁺ at m z of 285.0909 with an elemental analysis of C₂₀H₁₂O₂, which corresponds to a dihydroxybenzo[a]pyrene (dihydroxy-BaP) [Table 2], as reported elsewhere (de Llasera et al., 2016).

Dihydrodiol-BaP and BaP-quinone were also detected. A metabolite was observed at a retention time of 24.8 min with [M+1]⁺ at m z of 317.0811 that corresponds to C₂₀H₁₂04; it showed a base peak at m z of 299.0717 with elemental analysis of C₂₀H₁₁O₃ (M^{+.}-17, loss of OH), and a fragment at m z of 271.0763 with elemental analysis of C₁₉H₁₁O₂(M^{+.}-45, loss of CO₂H). These fragmentations are consistent with 4,5-chrysene-dicarboxylic acid and 4-(8-hydroxypyren-7-yl)-2-oxobut-3-enoic acid or its isomer 4-(7-hydroxypyren-8-yl)-2-oxobut-3-enoic acid (Table 2).

TABLE 2
High-resolution mass spectral data for BaP metabolites formed by Staphylococcus haemoliticus strain 10SBZ1A.

| | Observed | | | | Characteristics |
|---|---|---|---|---|---|
| | molecular | Retention | | | of major |
| | ion mass | time | Relative | Molecular | fragments |
| Metabolite | (calculated) | (min) | intensity | formula | (calculated) |
| Dihydroxy-BaP | 285.0909 | 22.92 | 41 | C₂₀H₁₂O₂ | C₂₀H₁₁O |
| | [M + 1] | | | | 267.0802 |
| | (285.0916) | | | | (267.0810) 73, |
| | | | | | C₁₉H₁₃O; |
| | | | | | 257.0954 |
| | | | | | (257.0966) 100 |
| BaP-quinone | 283.0756 | 26.55 | 93 | C₂₀H₁₀O₂ | C₁₉H₁₁O |
| | [M + 1] | | | | 255.0814 |
| | (283.0759) | | | | (255.0810) 100; |
| | | | | | C₁₈H₁₀, 226.0783 |
| | | | | | (226.0782) |
| 4,5-Chrysene- | 317.0811 | 24.89 | 9 | C₂₀H₁₂O₄ | C₂₀H₁₁O₃ |
| dicarboxylic acid or 4- | [M + 1] | | | | 299.0717 |
| (8-Hydroxypyren-7-yl)- | (317.0814) | | | | (299.0708) 100; |
| 2-oxobut-3-enoic acid | | | | | C₁₉H₁₁O₂ |
| or 4-(7-Hydroxypyren- | | | | | 271.0763 |
| 8-yl)-2-oxobut-3-enoic | | | | | (271.0759) 75. |
| acid | | | | | |
| 10- | 269.0607 | 24.87 | 71 | C₁₉H₁₀O₂ | C₁₉H₁₀O, |
| Oxabenzo[def]chrysene- | [M − 1] | | | | 254.0773 |
| 9-one or 7- | (269.0603) | | | | (254.0732) 43 |
| Oxabenzo[def]chrysene- | | | | | |
| 8-one | | | | | |
| 4-Formylchrysene-5- | 299.0716 | 26.84 | | C₂₀H₁₂O₃ | C₁₉H₁₁O |
| carboxylic acid | [M − 1] | | | | 255.0820 |
| | (299.0708) | | | | (255.0810) 100; |
| | | | | | C₁₈H₁₁ 227.0871 |
| | | | | | (227.0861) 9 |

A metabolite was detected at retention time of 24.87 min with [M−1]⁻ at m z 269.0607 with an elemental analysis of C₁₉H₁₀O₂, which corresponds to either 10-oxabenzo[def]chrysene-9-one or its isomer 7-oxabenzo[def]chrysene-8-one. This suggests that the detected dihydroxy-BaP could be 9,10-dihydroxy-BaP, which results in a ring opening at C9-C10, or 7,8-dihydroxy-BaP, which results in a ring opening at C7-C8; in both cases, a substituted pyrene is produced. A metabolite was observed at retention time of 26.84 min with [M−1]⁻ at m z 299.0716 with an elemental analysis of C₂₀H₁₂O₃, a base peak at m z of 255.0820 that corresponds to C₁₉H₁₁O₂ (M^{+.}-45, loss of CO₂H), and a fragment at m z of 227.0861 [C₁₈H₁₁, M^{+.}-73, loss of CO₂H and CO]. This fragmentation pattern is consistent with 4-formylchrysene-5-carboxylic acid, which indicates that the observed dihydroxy-BaP could be 4,5-dihydroxy-BaP.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

As referred to herein, all compositional percentages are by weight of the total composition, unless otherwise specified.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1: A method for degrading a polycyclic aromatic hydrocarbon (PAH), comprising:
contacting the polycyclic aromatic hydrocarbon or a composition comprising the PAH with a bacterium that is Staphylococcus haemolyticus or a bacterium having 16s rDNA that is at least 99% identical to the rDNA of Staphylococcus haemolyticus under conditions suitable for degradation of the polyaromatic hydrocarbon.

2. The method of claim 1, wherein the bacterium is Staphylococcus haemolyticus.

3: The method of claim 1, wherein the bacterium is Staphylococcus haemolyticus strain 10SBZ1A.

4. The method of claim 1, wherein the polycyclic aromatic hydrocarbon has five rings.

5. The method of claim 1, wherein the polycyclic aromatic hydrocarbon has four rings.

6. The method of claim 1, wherein the polycyclic aromatic hydrocarbon has three rings.

7. The method of claim 1, wherein the polycyclic aromatic hydrocarbon has two rings.

8. The method of claim 1, wherein the polycyclic aromatic hydrocarbon is selected from the group consisting of naphthalene (NAPH), phenanthrene (PHEN), anthracene (ANTH) and pyrene (PYR).

9. The method of claim 1, wherein the polycyclic aromatic hydrocarbon is benzo(a)pyrene (“BZP”).

10. The method of claim 1, wherein said contacting occurs in an aqueous medium having a salt content ≥5 wt. %.

11. The method of claim 1, wherein said contacting occurs in an aqueous medium having a salt content ≥10 wt. %.

12. The method of claim 1, wherein said contacting occurs in an aqueous medium having a salt content ≥15 wt. %.

13. The method of claim 1, wherein said contacting occurs at a temperature ranging from 20 to 45° C., at a salinity ranging from 5 to 15% wt/wt, and at a pH ranging from 6.5 to 7.5.

14. The method of claim 1, wherein said contacting occurs in the presence of a long chain fatty acid.

15. The method of claim 1, wherein said contacting occurs in the presence of glycerol.

16. An aqueous composition, comprising:
at least 5 wt % NaCl, benzo(a)pyrene (BZP), and
a Staphylococcus haemolyticus strain that grows in the presence of at least 5 wt. % NaCl and can utilize BZP as its sole carbon source.

17. The aqueous composition of claim 16 wherein the Staphylococcus haemolyticus strain grows in the presence of at least 10 wt. % NaCl and can utilize BZP as its sole carbon source.

18. The aqueous composition of claim 16, wherein the Staphylococcus haemolyticus strain grows in the presence of at least 15 wt. % NaCl and can utilize BZP as its sole carbon source.

19. A Staphylococcus haemolyticus strain that has been genetically or epigenetically modified to increase its growth rate in a medium containing at least 5 to 15 wt. % NaCl.

20. The Staphylococcus haemolyticus strain of claim 19 that is strain 10SBZ1A which has been genetically or epigenetically modified to increase its growth rate or ability to degrade a PAH compared to a parent strain 10SBZ1A in a medium containing at least 5 to 15 wt. % wt % NaCl.


SEQUENCE LISTING (4 sequences)

SEQ ID NO: 1 - DNA, 20 bp, Artificial Sequence
  Primer 1492F
agagtttgatcmtggctcag

SEQ ID NO: 2 - DNA, 22 bp, Artificial Sequence
  Primer 1492R
tacggytaccttgttacgactt

SEQ ID NO: 3 - DNA, 20 bp, Artificial Sequence
  Primer 518F
ccagcagccgcggtaatacg

SEQ ID NO: 4 - DNA, 18 bp, Artificial Sequence
  Primer 800R
taccagggtatctaatcc